(19) United States
(12) Patent Application Publication
(10) Pub. No.: US 2015/0087040 A1
(43) Pub. Date: Mar. 26, 2015

(54) PRODUCTION OF ETHANOL AND RECYCLE WATER IN A CELLULOSIC FERMENTATION PROCESS

(71) Applicant: E I DU PONT DE NEMOURS AND COMPANY, Wilmington, DE (US)

(72) Inventors: William D. Parten, Wilmington, DE (US); Rahul B. Kasat, Wilmington, DE (US)

(73) Assignee: E I DU PONT DE NEMOURS AND COMPANY, Wilmington, DE (US)

(21) Appl. No.: 14/037,407

(22) Filed: Sep. 26, 2013

(51) Int. Cl. C12P 7/10

(52) U.S. Cl. CPC C12P 7/10
USPC 435/165

(57) ABSTRACT

Processes for producing ethanol from fermentation systems employing cellulosic feedstocks are provided, where water is processed for recycle. The water handling portion of the process employs multi-effect evaporators where the first effect evaporator is maintained at temperatures in excess of 105° C., and the evaporation train, beer column form a heat integrated system.

### FIELD OF THE INVENTION

The present invention relates to processes for producing ethanol in a fermentative process from cellulosic feedstocks, and for processing water for recycle. Specifically, a train of at least four evaporation equivalents is heat integrated with a beer column and rectification column to provide efficient water handling.

### BACKGROUND OF THE INVENTION

Ethanol is an important source of energy and useful as an alternative to petroleum based gasoline and diesel products. Ethanol is produced by fermentation of a wide variety of organic feedstocks to provide a beer that is distilled and dehydrated to produce a high purity product. The majority of fuel ethanol today is produced from grain, starch or sugar based feedstocks. These methods typically include fermentation of a mixture of water and milled grain to yield alcohol, distillation of the fermented mixture to recover alcohol as a top product and distillery bottom by-products, which includes grain solids and thin stillage of dissolved solids in water. The distillary by-products are typically concentrated by evaporation of water therefrom, to yield Distiller's Dried Grains with Solubles (DDGS), a valuable feed for livestock.

Typical grain ethanol facilities generally have the following elements in common:
1) There is an absorption or scrubbing process on the vent stream from the fermenters to recover methanol, ethanol, higher alcohols and acetaldehyde from the co-produced carbon dioxide. This normally draws the makeup water into the plant and is typically a cold stream.
2) Distillation is employed to produce a stream that is concentrated in ethanol up to the azeotropic composition with water and a predominantly water and solids stream that is free of ethanol. This process is generally achieved by use of two distillation columns known as a Beer Column and a Rectifying Column
3) The azeotropic ethanol stream is then subject to further concentration via a two liquid phase azeotropic distillation involving an entrainer or more usually by concentration in molecular sieves to produce a fuel grade ethanol which is the prime product of the process.
4) The solids from the aqueous stream mentioned in 2) are separated via centrifugation or some other means and may be further dried to produce an animal feed or material for fuel.
5) A fraction of the aqueous stream (backset) resulting from 4) may be recycled to the front end of the plant to form a fraction of the feed to the fermenter whilst the remainder will be evaporated to remove impurities that would otherwise build up. These impurities include sugars that can't be fermented, proteins and salts and are purged as a concentrated liquid stream. The condensate from the evaporation may be recycled directly to the fermentation with the backset. The evaporation process is usually two or three stages and will be heat integrated with the distillation process.

Grain, starch and sugar based processes are becoming increasingly less desirable as they necessarily rely on a food source and have negatively impacted global food prices. Production of ethanol from cellulosic agricultural and other waste feedstocks avoids these problems. Cellulosic feedstocks are those that typically contain cellulose and hemicellulose, as well as lignin. Suitable feedstocks for the production ethanol from cellulosic feedstocks include biomass such as corn cob, corn stover, grasses, woody biomass, sugar cane bagasse, as well as industrial waste products containing a high cellulosic component. Processes for the generation of alcohols and particularly ethanol from cellulosic feedstocks are described in numerous publications (see for example Aden et al. in “Lignocellulosic Biomass to Ethanol Process Design and Economics Utilizing Co-Current Dilute Acid Prehydrolysis and Enzymatic Hydrolysis for Corn Stover”, NREL Report No. TP-510-32438; NREL Report NREL/TP-510-37092, “Plants from Corn Starch and Lignocellulosic Feedstocks (Revised)”; and Madson, P. W. and D. A. Monceaux, (Fuel Ethanol Production), in “Fuel Alcohol Textbook” Alltech Inc., 1999. Various aspects of a cellulosic ethanol process are disclosed in commonly owned U.S. Pat. No. 7,629,156, U.S. Pat. No. 7,666,282 U.S. Pat. No. 7,741,084, U.S. Pat. No. 7,741,119, U.S. Pat. No. 7,781,191, U.S. Pat. No. 7,803,623, U.S. Pat. No. 7,807,419, U.S. Pat. No. 7,819,976, U.S. Pat. No. 7,897,396, U.S. Pat. No. 7,910,338, U.S. Pat. No. 7,932,063, U.S. Pat. No. 7,989,206, U.S. Pat. No. 7,998,713, U.S. Pat. No. 7,998,722, U.S. Pat. No. 8,216,809, U.S. Pat. No. 8,241,873, U.S. Pat. No. 8,241,880, U.S. Pat. No. 8,247,208, U.S. Pat. No. 8,278,070, U.S. Pat. No. 8,304,213, and U.S. Pat. No. 8,304,535.

Processes for cellulosic biomass production of alcohols face certain challenges not present in typical grain ethanol plants. A major challenge is the consumption of an increased amount of water, requiring processes able to handle the increased water load. Increased water consumption arises from the reduced ethanol concentration in the fermentation broth, which is typically in the range of 5% to 10% ethanol vs 11% to 15% ethanol for grain based ethanol production. In addition, residual water that remains in a cellulosic process after both ethanol and solids have been removed from fermentation broth contains higher levels of impurities than does that from a grain process. The condensate that results from evaporation of this water contains significantly more acetic acid and cannot be recycled directly to fermentation. The water processing load in a cellulosic plant may be up to three times higher per unit of ethanol produced than is typical in a grain to ethanol plant, but still needs to be accommodated by a process that uses a comparable energy input to result in an acceptable energy footprint.

Thus simply transferring the methods of working that are established within the grain ethanol industry to a cellulosic ethanol process is not sufficient for an economically and energetically competitive cellulosic ethanol process. Hence cellulosic ethanol presents a new set of challenges, and processes that are able to meet those challenges should be considered against that background rather than in the context of a grain based process.

To make fuel grade alcohol production more economical from cellulosic feedstocks it is desirable to reduce the external energy and water required to operate the various steps in the alcohol production process. This can be achieved, for example, by maximal heat integration and recycling of waste water streams back into the process.

U.S. Pat. No. 7,572,353 and U.S. Pat. No. 7,297,236 disclose using first and second effect evaporators for concentrating thin stillage in a grain based process, where second effect steam is used as heat for distillation. The concept of using a three-stage evaporator, and the possibility of use of vapor recompression of low quality waste steam, in a cellulosic ethanol process are discussed generally by Ryan M Melsert (“Energy Optimization Of The Production Of Cellulosic Ethanol From Southern Pine”, Master's Thesis, University of Georgia, Nov. 13, 2007), where the first effect evaporator is run at 83.4° C.

There remains a need to develop an effective heat integrated distillation/evaporation system for a cellulosic ethanol fermentation process that can handle up to three times the amount of water used in a grain ethanol system with energy efficiency, and that recycles water.

### SUMMARY OF THE INVENTION

Production of fuel ethanol from cellulosic fermentations faces challenges in energy efficiency and water load not seen in comparable grain ethanol processes. In order to increase energy and water handling efficiencies applicants provide a system with heat integration and high water handling capacity made possible by Applicants' finding that heat exchange surfaces are not fouled with prolonged high temperature operation in a cellulosic ethanol process.

Accordingly the invention provides a process for the production of ethanol and recycle water comprising:
a) providing a hydrolysate fermentation medium;
b) fermenting the medium in a fermenter in the presence of a microorganism that produces ethanol to produce a beer comprising from about 6% to about 10% ethanol, water, solutes, and solids;
c) providing a distillation and water handling system comprising the elements:
   I) a beer column;
   II) a rectification column;
   III) a solids removal mechanism; and
   IV) an evaporation train comprising at least four evaporation effect equivalents;
wherein each element in the system is connected to at least one other element in the system, and wherein the beer column, the rectification column, and the evaporation train form a heat integrated system;
d) passing the beer into the beer column of (I) where a beer column ethanol-rich vapor stream is separated from a water stream containing solutes and solids;
e) condensing the beer column ethanol-rich vapor stream forming a beer column ethanol-rich stream;
f) passing the beer column ethanol-rich stream into the rectification column of (II) wherein a further ethanol-enriched rectification column vapor stream is separated from an ethanol depleted water stream;
g) passing the water stream containing solutes and solids of (d) through the solids removal mechanism of (III) producing a water stream containing solutes with solids substantially removed;
h) passing the water stream containing solutes with solids substantially removed of (g) through the evaporation train of (IV) producing an evaporate comprising water containing high-volatility solutes and a syrup comprising low-volatility solutes, wherein a first effect evaporator of the evaporator train of (IV) is run at a temperature that is greater than 105° C. for at least about 10 days without fouling of heat exchange surfaces; and
i) treating the evaporate of (h) to substantially remove the high-volatility solutes producing recycle water;
wherein ethanol-enriched vapor is produced and water is processed for recycle.
   In some embodiments wherein the at least four evaporation effect equivalents comprises at least one first evaporator stage and at least one vapor recompression unit.

### BRIEF DESCRIPTION OF THE FIGURES

FIG. 1 is a schematic diagram of a cellulosic ethanol process flow sheet.

FIG. 2 is a graph of the temperatures of the three effects of evaporators with measurements over 12 days.

FIG. 3 is a graph of the heat transfer coefficients for the three effects of evaporators with calculations made for the run over 11 days.

### DETAILED DESCRIPTION OF THE INVENTION

The following definitions and abbreviations are to be use for the interpretation of the claims and the specification.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “consists of,” or variations such as “consist of” or “consisting of,” as used throughout the specification and claims, indicate the inclusion of any recited integer or group of integers, but that no additional integer or group of integers may be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

“Stripping” as used herein means the action of transferring all or part of a volatile component from a liquid stream into a gaseous stream.

“Rectifying” as used herein means the action of transferring all or part of a condensable component from a gaseous stream into a liquid stream in order to separate and purify lower boiling point components from higher boiling point components.

The term “low-volatility solutes” refers to components that in the evaporation process will largely stay in the remaining liquid or syrup from the evaporation process. Examples of low-volatility solutes include high boiling organics such as sugars and inorganic soluble minerals such as sodium, potassium and nitrate or sulphate ions.

The term “high-volatility solutes” refers to components that will largely follow the water being evaporated and so distribute between the evaporator condensate and the remaining liquid or syrup. Examples of high-volatility solutes include components such as ethanol and acetic acid, the majority of which will partition into the condensate from an evaporation process.

The term “lignocellulosic” refers to a composition comprising both lignin and cellulose. Lignocellulosic material may also comprise hemicellulose.

The term “cellulosic” refers to a composition comprising cellulose and additional components, including hemicellulose. A cellulosic composition may also include lignin.

The term “saccharification” refers to the production of fermentable sugars from polysaccharides.

The term “fermentable sugar” refers to oligosaccharides and monosaccharides that can be used as a carbon source by a microorganism in a fermentation process.

The term “pretreated biomass” means biomass that has been subjected to pretreatment prior to saccharification.

The term “lignocellulosic biomass” refers to any lignocellulosic material and includes materials comprising cellulose, hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Lignocellulosic biomass includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat straw, barley straw, hay, rice straw, switchgrass, waste paper, sugar cane bagasse, sorghum plant material, soybean plant material, components obtained from milling of grains, trees, branches, roots, leaves, wood chips, sawdust, shrubs and bushes, vegetables, fruits, and flowers.

The term “hydrolysate” refers to the product resulting from saccharification of biomass. The biomass may also be pretreated or pre-processed prior to saccharification.

The term “biomass hydrolysate fermentation broth” is broth containing product resulting from biocatalyst growth and production in a medium comprising biomass hydrolysate. This broth includes components of biomass hydrolysate that are not consumed by the biocatalyst, as well as the biocatalyst itself and product made by the biocatalyst.

The term “evaporation effect equivalent” refers to a unit of evaporation that occurs in a single evaporator stage from an external heat source. Thus, for example, four single stage evaporators where steam produced from the first stage provides a heat source for the second stage, steam from the second stage provides a heat source for the third stage, and steam from the third stage provides a heat source for the fourth stage, would provide four evaporation effect equivalents. A vapor recompression unit attached to a single stage evaporator can allow one evaporator to achieve one or more additional units of evaporation. For example, if a single evaporation unit were supplied a unit of heat external to it and the steam it produced were compressed and supplied back to the same evaporator as an additional heat source, it may be possible to achieve two, three, four or more times the amount of evaporation than could have been achieved by use of only the initial external heat source. Hence the vapor recompression unit provides a multiplier to the “number of effects of evaporation” based on the amount of steam that is compressed to achieve further evaporation versus the amount of steam that would have been generated only by use of the initial external heat source. Thus, for example, a single stage evaporator or double stage evaporators, in conjunction with one or more vapor recompression units, can provide four evaporation effect equivalents.

The present invention provides for ethanol production from cellulosic biomass, and production of recycle water from ethanol production process water. In the present process there is energy efficiency and increased water evaporation capacity. As compared with processes for the production of fuel ethanol from grain or sugar fermentations, there is a need to evaporate up to as much as 3-fold more water in cellulosic biomass hydrolysate based ethanol production systems. This need stems from the lower ethanol concentration in the beer that is produced from the cellulosic biomass hydrolysate fermentation process when compared to the grain fermentation process, and the fact that the fraction of water that can be recycled directly without first passing through an evaporation process is very much less for the cellulosic process than is the case for a grain based process.

### Biomass Hydrolysate

The present process is a cellulosic ethanol process in which medium used in fermentation contains hydrolysate prepared from cellulosic biomass, which is a hydrolysate fermentation medium. The biomass used may be any cellulosic or lignocellulosic material, for example, bioenergy crops, agricultural residues, municipal solid waste, industrial solid waste, yard waste, wood, forestry waste and combinations thereof. Cellulosic biomass hydrolysate is produced by saccharification of cellulosic (including lignocellulosic) biomass. Typically the biomass is pretreated prior to saccharification. Biomass may be treated by any method known by one skilled in the art to produce fermentable sugars in a hydrolysate. Typically the biomass is pretreated using physical and/or chemical treatments, and saccharified enzymatically. Physical and chemical treatments may include grinding, milling, cutting, base treatment such as with ammonia or NaOH, and/or acid treatment. Particularly useful is a low ammonia pretreatment where biomass is contacted with an aqueous solution comprising ammonia to form a biomass-aqueous ammonia mixture where the ammonia concentration is sufficient to maintain an alkaline pH of the biomass-aqueous ammonia mixture but is less than about 12 wt. % relative to dry weight of biomass, and where dry weight of biomass is at least about 15 wt % solids relative to the weight of the biomass-aqueous ammonia mixture, as disclosed in commonly owned U.S. Pat. No. 7,932,063, which is herein incorporated by reference.

Enzymatic saccharification of cellulosic or lignocellulosic biomass typically makes use of an enzyme composition or blend to break down cellulose and/or hemicellulose and to produce a hydrolysate containing sugars such as, for example, glucose, xylose, and arabinose. Saccharification enzymes are reviewed in Lynd, L. R., et al. (Microbiol. Mol. Biol. Rev., 66:506-577, 2002). At least one enzyme is used, and typically a saccharification enzyme blend is used that includes one or more glycosidases. Glycosidases hydrolyze the ether linkages of di-, oligo-, and polysaccharides and are found in the enzyme classification EC 3.2.1.x (Enzyme Nomenclature 1992, Academic Press, San Diego, Calif. with Supplement 1 (1993), Supplement 2 (1994), Supplement 3 (1995, Supplement 4 (1997) and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650 1999, respectively]) of the general group “hydrolases” (EC 3.). Glycosidases useful in the present method can be categorized by the biomass components they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), or feruloyl esterases (EC 3.1.1.73) to promote the release of polysaccharides from other components of the biomass. It is known in the art that microorganisms that produce polysaccharide-hydrolyzing enzymes often exhibit an activity, such as a capacity to degrade cellulose, which is catalyzed by several enzymes or a group of enzymes having different substrate specificities. Thus, a “cellulase” from a microorganism may comprise a group of enzymes, one or more or all of which may contribute to the cellulose-degrading activity. Commercial or non-commercial enzyme preparations, such as cellulase, may comprise numerous enzymes depending on the purification scheme utilized to obtain the enzyme. Many glycosyl hydrolase enzymes and compositions thereof that are useful for saccharification are disclosed in WO 2011/038019. Additional enzymes for saccharification include, for example, glycosyl hydrolases that hydrolyze the glycosidic bond between two or more carbohydrates, or between a carbohydrate and a noncarbohydrate moiety.

Saccharification enzymes may be obtained commercially. Such enzymes include, for example, Spezyme® CP cellulase, Multifect® xylanase, Accelerase® 1500, and Accellerase® DUET (Danisco U.S. Inc., Genencor International, Rochester, N.Y.), and Novosyme-188 (Novozymes, 2880 Bagsvaerd, Denmark). In addition, saccharification enzymes may be unpurified and provided as a cell extract or a whole cell preparation. The enzymes may be produced using recombinant microorganisms that have been engineered to express one or more saccharifying enzymes. For example, the H3A protein preparation used herein for saccharification of pretreated cellulosic biomass is an unpurified preparation of enzymes produced by a genetically engineered strain of Trichoderma reesei, which includes a combination of cellulases and hemicellulases and is described in WO 2011/038019, which is incorporated herein by reference.

Fermentation media containing biomass hydrolysate may contain a percent of hydrolysate with one or more additional sugars and/or other added components, or the media may contain 90% or more hydrolysate with minor additions. To improve growth, sorbitol, mannitol, or a mixture thereof may be included in the medium as disclosed in commonly owned U.S. Pat. No. 7,629,156, which is incorporated herein by reference. Typically a final concentration of about 5 mM sorbitol or mannitol is present in the medium. In various embodiments cellulosic biomass hydrolysate is at least about 50%, 60%, 70%, 80%, 90% or 95% of the final volume of fermentation broth. Typically about 10% of the final volume of fermentation broth is seed inoculum containing the biocatalyst.

The solids content of biomass hydrolysate is typically between about 10% and 40%, depending on the pretreatment and saccharification methods employed. More typically the solids content is about 25%, with a medium containing 90% cellulosic biomass hydrolysate having about 23% solids.

### Fermentation

In the present process the medium comprising hydrolysate is fermented in a fermenter, which is any vessel that holds the hydrolysate fermentation medium and biocatalyst, and has valves, vents, and/or ports used in managing the fermentation process. In the present process the biocatalyst is a microorganism that produces ethanol. The microorganism may naturally produce ethanol, or be genetically engineered to produce ethanol, or to have improved ethanol production. Any of these microorganisms is an ethanologen. In one embodiment the ethanologen is a yeast or a bacterium. In one embodiment the yeast is of the genus Saccharomyces. In one embodiment the bacterium is of the genus Zymomonas or Zymobacter.

The biocatalyst may be engineered to have improved ethanol production in hydrolysate medium. The biocatalyst may be engineered for xylose utilization such as in Saccharomyces cerevisiae which is described in Matsushika et al. (Appl. Microbiol. Biotechnol. (2009) 84:37-53) and in Kuyper et al. (FEMS Yeast Res. (2005) 5:399-409). The biocatalyst may be engineered for xylose utilization such as in Zymomonas mobilis which is described in U.S. Pat. No. 5,514,583, U.S. Pat. No. 5,712,133, U.S. Pat. No. 6,566,107, WO 95/28476, Feldmann et al. ((1992) Appl Microbiol Biotechnol 38: 354-361), and Zhang et al. ((1995) Science 267:240-243). Examples of strains engineered to express a xylose utilization metabolic pathway include CP4(pZB5) (U.S. Pat. No. 5,514,583), ATCC31821/pZB5 (U.S. Pat. No. 6,566,107), 8b (US 20030162271; Mohagheghi et al., (2004) Biotechnol. Lett. 25; 321-325), and ZW658 (ATTCC # PTA-7858). The biocatalyst may be engineered for arabinose utilization as described in U.S. Pat. No. 5,843,760, and US 2011/0143408, which are incorporated herein by reference. Examples of additional modifications that may be engineered in Zymomonas include reduced expression of the endogenous himA gene as described in U.S. Pat. No. 7,897,396, which is incorporated herein by reference; reduced glucose-fructose oxidoreductase (GFOR) activity as described in U.S. Pat. No. 7,741,119, which is incorporated herein by reference; increased ribose-5-phosphate isomerase (RPI) activity, as disclosed in commonly owned and co-pending US 20120156746, which is incorporated herein by reference; expression of xylose isomerase as part of the xylose utilization metabolic pathway using a mutant, highly active promoter as disclosed in U.S. Pat. No. 7,989,206 and U.S. Pat. No. 7,998,722, which are incorporated herein by reference; expression of a Group I xylose isomerase as disclosed in commonly owned and co-pending US 2011-0318801, which is incorporated herein by reference; and adaptation of a strain for growth in stress culture containing ethanol and ammonium acetate as disclosed in US 2011-0014670, which is incorporated herein by reference.

Fermentation is carried out with conditions appropriate for the particular biocatalyst used. Adjustments may be made for conditions such as pH, temperature, oxygen content, and mixing. Conditions for fermentation of yeast and bacterial biocatalysts are well known in the art.

In addition, saccharificatoin and fermentation may occur at the same time in the same vessel, called simultaneous saccharification and fermentation (SSF). In addition, partial saccharification may occur prior to a period of concurrent saccharification and fermentation in a process called HSF (hybrid saccharification and fermentation).

For large scale fermentations, typically a smaller culture of the biocatalyst is first grown, which is called a seed culture. The seed culture is added to the fermentation medium as an inoculum typically in the range from about 2% to about 20% of the final volume.

Typically fermentation by the biocatalyst produces a beer containing from about 6% to about 10% ethanol. The beer may contain between about 7% and about 9% of ethanol. In addition, the beer contains water, solutes, and solids from the hydrolysate medium and from biocatalyst metabolism of sugars in the hydrolysate medium. In particular, the beer contains acetaldehyde in levels that are higher than those found in a beer produced from grain fermentation. In addition, when ammonia is used for pretreatment of the biomass prior to saccharification producing hydrolysate used in fermentation media, ammonia is present in the beer. These contaminants have high volatility and will co-purify with the ethanol product during distillation.

### Ethanol Purification

Beer produced from biomass hydrolysate fermentation, which contains ethanol, water, solutes, and solids, is passed to a beer column where an ethanol-rich vapor stream is separated from a water stream containing solutes and solids, also called whole stillage. Typically solids are separated from the beer column water stream using a filter press, centrifugation, or other solid separation method. The remaining water containing solutes, also called thin stillage, is passed through an evaporation train to produce a syrup, containing low-volatility solutes, and water vapor, containing high-volatility solutes, that may be condensed and further treated to remove contaminants, then recycled. Treatment may be using an anaerobic digester. Use of anaerobic digesters is well known by one skilled in the art for bacterial hydrolysis of organic materials, and typically production of methane and carbon dioxide. This biogas may be used directly as fuel, or upgraded to higher quality biomethane fuel. The evaporation train is described further below.

The beer column ethanol-rich vapor stream is typically about 30%-55% ethanol by volume. The ethanol-rich vapor stream is condensed and passed to a rectification column where a further ethanol-enriched rectification column vapor stream is produced, as well as an ethanol depleted water stream. The further ethanol-enriched rectification column vapor stream is typically about 90 to 95% ethanol by volume, which is close to the ethanol/water azeotrope (95.63% ethanol and 4.37% water, by weight). This stream is super-heated and passed to a molecular sieve for further water removal producing a molecular sieve ethanol product, which is a further purified ethanol product. This ethanol product is about 99% ethanol by volume.

The condensed molecular sieve ethanol product is typically the final ethanol product in a grain ethanol process. The corresponding molecular sieve ethanol product in a cellulosic ethanol process, where biomass hydrolysate is fermented, contains levels of contaminants not found in the grain ethanol product. Management of these contaminants needs to be addressed in the cellulosic ethanol process. Specifically, applicants have measured acetaldehyde in the molecular sieve ethanol product from a hydrolysate fermentation process and found the level to be higher than the 200 to 500 ppm typically found in a grain ethanol molecular sieve ethanol product.

To reduce the amount of acetaldehyde in the final ethanol product, the molecular sieve ethanol product may be passed through a product distillation column. Distillation in this column is carried out so that acetaldehyde, ammonia and carbon dioxide are concentrated overhead, and the bottoms stream is the final ethanol product. The operating pressure of the distillation column may be linked to that of the molecular sieve unit so that material flows to the column by pressure difference. The operating pressure may also be high enough so that reflux can be returned to the column by use of a condenser utilizing cooling water for heat removal, such that there are minimal ethanol losses overhead. The overheads ethanol composition may be less than 50%, less than 30% or preferably less than 15% ethanol. The molecular sieve alcohol product stream may be passed through a condenser or partial condenser prior to passing it through the product distillation column.

The ethanol product from the product distillation column is the final ethanol product. This product contains reduced levels of acetaldehyde in comparison to the molecular sieve ethanol product. Typically the final alcohol product contains less than about 800 ppm of acetaldehyde. The final alcohol product may contain less than 800 ppm, 700 ppm, 600 ppm, 500 ppm, 400 ppm, or 300 ppm of acetaldehyde. In addition, the final ethanol product typically contains reduced levels of other contaminants such as carbon dioxide and ammonia, in comparison to the molecular sieve ethanol product. Typically the final ethanol product contains less than about 10 ppm of CO₂, and less than about 1 ppm of ammonia.

A contaminant stream is produced from the product distillation column. This stream is treated to avoid release of acetaldehyde and other contaminants to the atmosphere. The stream may be treated by any method known by one skilled in the art for removing the contaminants, such as acetaldehyde, CO₂, and/or ammonia. In various embodiments the product distillation column contaminant stream is treated in a boiler, a catalytic converter, a catalytic oxidizer, a thermal oxidizer, or in any combination of these units.

### Rectification and Scrubber Water Recycle

With inclusion of the product distillation column that removes contaminants from the molecular sieve ethanol product, an increased load of acetaldehyde and other contaminants can be processed through the rectification column without affecting the final ethanol product. A water stream containing acetaldehyde and other contaminants results from passing vapor from a fermentation vent stream through a scrubber. The scrubber water stream from the fermentation vapor scrubber may be passed to the rectification column. This stream contains acetaldehyde and carbon dioxide from the fermenter, as well as some ethanol. In addition, the stream contains ammonia if biomass was pretreated with ammonia during preparation of hydrolysate used in the fermentation medium. The scrubber water stream enters the rectification column below the feed from the beer column, because it has a reduced level of ethanol compared to the beer column overheads, but sufficiently high up the rectification column to facilitate removal of ammonia, carbon dioxide and acetaldehyde from the bottom of the rectification column.

In addition, rather than using fresh makeup water in the fermentation vapor scrubber as is typical in a grain ethanol process, use of fresh water can be reduced in this aspect of the cellulosic ethanol process by using ethanol depleted water from the rectification column in the fermentation vapor scrubber. At least a portion of the ethanol depleted rectification column water stream may be passed to the fermentation vapor scrubber. Thus the ethanol depleted rectification column water and fermentation vapor scrubber water may be used in a water recycle loop between the rectification column and fermentation vapor scrubber. These process waters may be used in the recycle loop without additional purification steps. The temperature at which water is required in the fermentation vapor scrubber is less than that of the water that exits the rectification column, so typically heat is interchanged between the water feed to the scrubber and the water returning from the scrubber to the rectification column in a process to process exchanger, with the final cooling of the water feed to the scrubber being accomplished using an exchanger where the utility stream is cooling water or chilled water.

In this recycle process, a large amount of water can be used on the scrubber to ensure more efficient capture of the acetaldehyde that enters in the fermentation vent vapor. A portion of the carbon dioxide will also be captured in the water exit stream from the scrubber, rather than being released to atmosphere as is typical. When using this recycle process, a greater amount of acetaldehyde and carbon dioxide will be present in the further ethanol-enriched rectification column vapor stream, which will pass to the molecular sieve. A majority of the acetaldehyde and carbon dioxide will pass through the molecular sieve with the product ethanol. At least a portion of the acetaldehyde and carbon dioxide are removed from the product by the product distillation column, reducing the levels to levels that are comparable to those present in a grain ethanol product, or lower. Contaminant levels in the product are typically as described above.

### Process Diagram

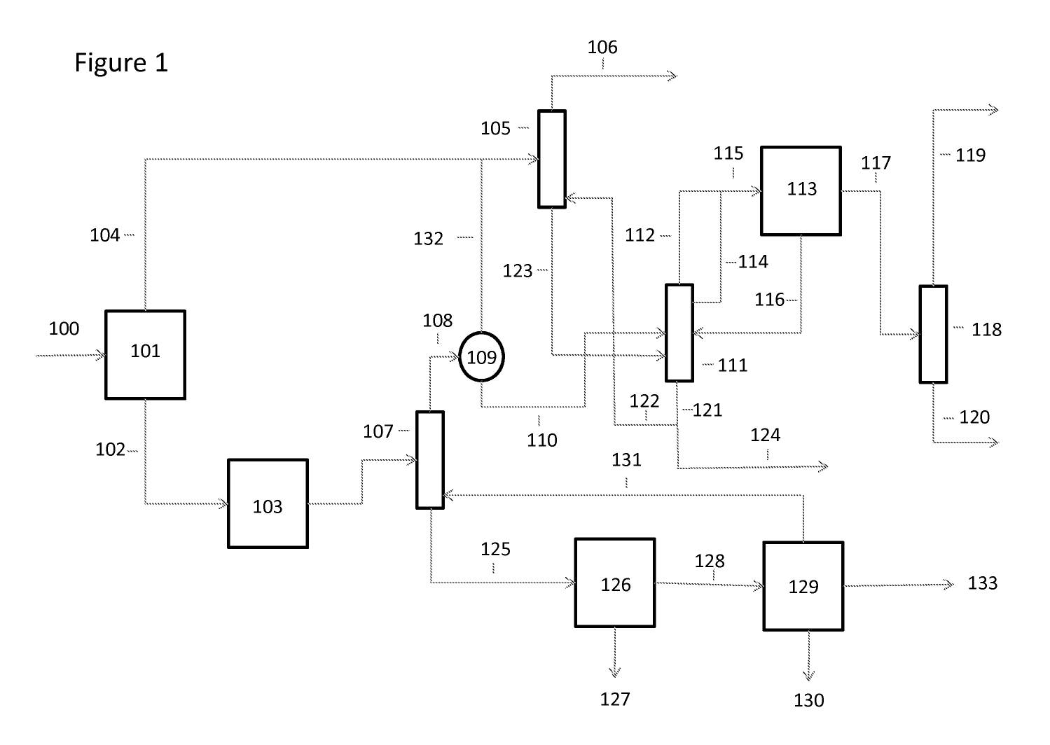

A schematic diagram in FIG. 1 shows a flow sheet representing an example of process stages for a cellulosic ethanol process from the entry of feed (100) into the fermenter (101) through production of the bottoms stream (120) which is the final ethanol product from the product column (118). The feed to the fermenter includes fermentation medium containing cellulosic biomass hydrolysate and biocatalyst inoculum, which are either mixed or added separately to the fermenter.

With reference to FIG. 1, a beer flow (102) from the fermenter (101) passes to an interim storage vessel, the beer well (103). Vent gases given off in fermentation, which are principally carbon dioxide (CO₂), form a vent gas flow (104) which passes to a fermentation vapor scrubber, also called a CO₂ scrubber, (105) for recovery of ethanol and acetaldehyde. A CO₂ vent stream (106) passes to the atmosphere. Beer from the beer well is passed to a beer column (107) where ethanol with water from the beer is removed in a vapor overheads product (108; a beer column ethanol-rich vapor stream), with the remainder of the beer forming a liquid and solid stream, called whole stillage, (125) that is substantially free of ethanol.

The beer column vapor overheads product flow (108) passes to a beer column condenser (109) producing a small vent stream (132) which passes to the fermentation scrubber (105) and a liquid overheads stream (110). The resulting beer column liquid overheads product condensate flow (a beer column ethanol-rich stream) (110) feeds a rectification column (111). In the rectification column there is further distillation and a rectification column overheads flow (a further ethanol-enriched rectification column vapor stream) (112) is superheated and passes to a molecular sieve unit (113) to further remove water from the ethanol stream. Also a side stream vapor product flow (114) which contains fusel oils is taken from an appropriate location of the rectification column, is combined with the rectification column overheads flow, the mixture (115) is superheated, and is passed to the molecular sieve unit (113) to further remove water from the ethanol stream. Thus fusel oils are combined with ethanol in the eventual product from the process. A molecular sieve purge (116) flows from the molecular sieve to the rectification column. This stream may instead flow to the beer column or the beer well. A dry ethanol flow (a molecular sieve ethanol product stream) (117) from the molecular sieve is passed to a product distillation column (118) where contaminants such as acetaldehyde, ammonia, and carbon dioxide are removed in a purge stream (119) and the bottoms stream (120) is the final ethanol product.

From the rectification column bottom an ethanol depleted rectification column water stream (121) exits, and a portion of this stream (122) is cooled and passed to the fermentation vapor scrubber (105) as scrubbing water. This water absorbs ethanol and acetaldehyde in the scrubber. The fermentation vapor scrubber bottom stream flow (a scrubber water stream) (123) is returned with appropriate heat interchange as a second feed to the rectification column (111) thereby recovering ethanol and acetaldehyde for further processing. Thus there is a recycle loop of water streams between the fermentation vapor scrubber and the rectification column.

The remaining ethanol depleted rectification column water stream (124) is typically heat interchanged with the feed to the rectification column (110) and is then passed for further treatment using an anaerobic digester or other purification technique before being recycled in the process as process water.

The whole stillage (125) is further processed by a solids removal mechanism such as a filtration unit (126) to remove solids producing a filter cake (127). The separated liquid flows as thin stillage (128) to an evaporation train (129) and the final evaporate condensate (130) is treated and used as clean recycle water in the cellulosic ethanol production process. A syrup stream (133) containing low volatility dissolved material is also produced from the evaporator train. Treating of the evaporate is to remove high-volatility solutes and may be by any known method such as anaerobic digestion, aerobic digestion, membrane separations, including nanofiltration, ultrafiltration and/or reverse osmosis separately or integral to aforementioned biotreatment alternatives, and ion exchange separation. In this depiction the beer column (107) is heat integrated with the evaporation train by injection of steam (131) from the evaporators.

### Distillation and Water Handling System

In the present process the large water load of a cellulosic ethanol process is managed using an evaporation train having at least four evaporation effect equivalents. Effective use of four or more evaporation effect equivalents is made possible due to the new finding that heat exchange surfaces may be maintained at temperatures above 105° C. for prolonged periods of time without fouling during processing of water used in the described cellulosic ethanol process. In these experiments, beer resulting from fermentation of biomass hydrolysate by an ethanologen was treated first in a beer column. In the beer column an ethanol-rich vapor stream was separated from a water stream containing solutes and solids. Following removal of solids, the remaining water stream containing solutes with solids substantially removed was passed to an evaporator that was maintained at 125° C. for a period of ten days. The heat exchange surfaces of the evaporator remained sufficiently clean that heat transfer performance was sufficient to allow efficient evaporation, indicating the heat exchange surfaces were not fouled.

Hydrolysate produced by pretreatment and saccharification of lignocellulosic biomass is typically a slurry containing components of the biomass that are not broken down during pretreatment and saccharification, and that are not metabolizable by an ethanologen. Even with solids substantially removed from the beer, the remaining components from a hydrolysate used in fermentation could foul heat exchange surfaces as is common for a corresponding water stream in grain ethanol processes when high temperatures are used. The experimental finding that heat exchange surfaces at high temperatures (greater than 105° C.) are not substantially fouled by the water stream from the beer column, after substantially removing solids, allows the present scheme for ethanol production and recycle water processing with its heat integrated system to be operable for extended time periods.

In the present process the distillation and water handling system comprises a beer column, a rectification column, a solids removal mechanism, and an evaporation train including at least four evaporation effect equivalents. Each element in the system is connected to at least one other element in the system as shown in FIG. 1, which is described above. The beer column, the rectification column, and the evaporation train form a heat integrated system.

The water stream containing solutes and solids entering the distillation and water handling system is from the bottom of the beer column and first is treated with the solids removal mechanism. Solids may be removed by any known method such as by centrifugation or filtering. Solids are substantially removed from the water stream wherein less than about 5% of the original amount of solids remains in the liquid fraction. This water stream containing solutes is passed through the evaporation train, first entering a first effect evaporator that is run at a temperature that is greater than 105° C. The first effect evaporator is run at a temperature that is greater than 105° C. for at least about 10 days. The first effect evaporator may be run at a temperature that is greater than 105° C. for 10 days, 15 days, 20 days, or longer.

As described above, running the first effect evaporator at greater than 105° C. for at least 10 days is made possible by the discovery that the water stream containing solutes that is separated from beer produced in a cellulosic ethanol fermentation does not foul heat exchange surfaces at temperatures greater than 105° C. when run for a period of 10 days. The first effect evaporator may be run at a temperature of about 106° C., 110° C., 115° C., 120° C., or 125° C. or any temperature in-between. In addition, the ethanol depleted water stream from the rectification column, which may be passed to the evaporation train, does not cause substantial fouling of heat exchange surfaces at these temperatures.

Running the first effect evaporator at a temperature greater than 105° C. allows the evaporation train to use at least four evaporation effect equivalents in an economically viable process to handle the high water load in the cellulosic ethanol process. In addition, running the first effect evaporator at a temperature greater than 105° C. allows higher temperatures and pressures to be used in the additional evaporation equivalents to accommodate the high solute load from the hydrolysate fermentation beer, which causes boiling point elevation.

In one embodiment, four evaporation effect equivalents are a series of four in-line separate evaporators in a multi-effect train. The steam from each evaporator is used to run the subsequent in-line evaporator, with temperature and/or pressure decreasing in each subsequent in-line evaporator. In various embodiments, five or six evaporation effect equivalents are a series of five or six in-line separate evaporators in a multi-effect train. To drive evaporation, temperature differences between higher pressure condensing vapor and lower pressure material to be evaporated in each successive evaporator is at least 5° C. for economical operation, and preferably up to 10° C. Thus starting at a temperature that is greater than 105° C. allows economical use of four or more in-line evaporators, as opposed to running the first effect evaporator at a temperature below 105° C. as in a grain ethanol process, where at most three effect evaporators can be run economically. Typically each successive evaporator is run at lower temperature and pressure than the previous evaporator.

In one embodiment, four evaporation effect equivalents are at least a first evaporator stage and at least one vapor recompression unit that provides sufficient evaporator enhancement to be equivalent to four evaporation effects. In one embodiment there are two evaporator stages in a multi-stage train and at least one vapor recompression unit that provides sufficient enhanced evaporation capacity to provide evaporation capacity equivalent to four evaporation effects. In various embodiments there are one, two, three, four, or five evaporators and one, two, three, four, or five vapor recompression units, in any combination of number of evaporator stages and vapor recompression units where the number of vapor recompression units is equal to or less than the number of stages of evaporation, by which the final number of evaporation effects achieved exceeds the number that would have been provided by the evaporation stages alone.

In any of the combinations of evaporator stages and vapor recompression units making at least four evaporation effect equivalents, heat from the rectification column is used to heat the first evaporator stage. A first portion of heat from steam produced in the first evaporator stage is passed through the vapor recompression unit and back to the first evaporator stage, a second portion of heat from the steam produced in the first evaporator stage is passed either a) to a second evaporator stage if present, and heat from steam produced in the second stage evaporator is passed to the beer column, or b) directly to the beer column. Alternatively, there may be a recompression unit associated with the second evaporator stage and not the first evaporator stage.

In any of these embodiments the first evaporator stage is a first effect evaporator that is run at greater than 105° C. The vapor recompression unit, which is associated with an evaporator stage, compresses at least a part of the vapor produced from the evaporator with which it is associated and returns the compressed vapor to the same evaporator thereby providing additional heat. The additional heat provides additional evaporation effect equivalents compared to the single evaporator stage, based on the amount of steam that is compressed compared to the amount of evaporation that would have taken place based on the external heat supply alone.

An evaporator and vapor recompression system as a whole provides an evaporation capacity equivalent to a certain number of equivalents based on the sum of the original heat supplied plus the heat returned through vapor recompression divided by the original heat supplied. Where vapor recompression is applied to several evaporators in series, the number of equivalent stages is the product of the original heat supplied and the number of evaporators plus the product of the heat transferred through vapor recompression and the number of evaporators over which it is transferred, all divided by the original amount of heat supplied. Thus in various embodiments, four evaporation effect equivalents may be achieved using one or more evaporators and one or more vapor recompression units.

In any of the described evaporation trains each evaporator stage may be one evaporator unit or it may be a group of two or more evaporator units. The number of evaporator units for each effect evaporator may depend on the capacity needed, as well as the need to take an individual evaporator off-line for maintenance. For example, U.S. Pat. No. 7,297,236 describes providing valves on the various lines leading to the evaporators so that any one of multiple units in an effect evaporator can be taken off-line and by-passed for maintenance.

### Recycle Water

Evaporate produced by passing the water stream containing solutes with solids substantially removed through the evaporation train contains high-volatility solutes. These are solutes that partition to the evaporating liquid at temperatures of the evaporation train. In the present process in which biomass hydrolysate is used in the fermentation medium, a higher solute load is present in comparison to that in the evaporate from a grain ethanol process. Acetic acid is the most abundant high-volatility solute in the evaporate from the present process. The presence of high levels of acetic acid in the evaporate make it unusable for recycle at this stage. In the present process the evaporate containing high-volatility solutes is condensed and treated to substantially remove the high-volatility solutes, producing recycle water. The condensed evaporate may be treated by any method known to remove solutes. In various embodiments treating is by anaerobic digestion, aerobic digestion, membrane separations, including nanofiltration, ultrafiltration and/or reverse osmosis separately or integral to aforementioned biotreatment alternatives, or ion exchange separation, which are methods well known to one skilled in the art.

After this treatment the resulting water is clean enough to be recycled in the ethanol production process, and is called recycle water. At least a portion of the water is mixed with biomass or biomass hydrolysate and forms a part of the hydrolysate fermentation medium. In addition, the recycle water may be used in the biomass pretreatment section of the process.

### Heat Integration System

In the present process the beer column, the rectification column, and the evaporation train form a heat integrated system. Heat is cycled among these components to maximize re-use of heat and minimize introduction of heat in the present ethanol production process.

The heat required to run the rectification column depends on the approach to the azeotropic composition that is desired. In any case the heat required is governed by the rectification section of this column. The heat required to run the beer column is governed by the need to obtain a bottoms stream that is largely free of ethanol and so is governed by the stripping section of this column. The heat required to run the rectification column then is largely governed by the amount of ethanol being made and is largely independent of the concentration of ethanol in the beer. The heat required to run the beer column is dependent on the amount of ethanol made, but is also inversely dependent on the concentration of ethanol in the beer. At concentrations of ethanol in the beer above about 10%, the heat required to run the rectification column exceeds that required to run the beer column, at concentrations below 10% the heat required to run the beer column exceeds that required to run the rectification column. The precise number at which this balance point is reached depends on how close to the azeotropic concentration the process is run and at what pressures the separations are to be carried out, but the heat required in the distillation section of a grain to ethanol plant is generally governed by the needs of the rectification column while that in a cellulosic ethanol plant is governed by that of the beer column, unless to provide a convenient number of evaporation stages the needs in the evaporation stages exceed those for the beer column.

Thus in the present process with ethanol concentration in the fermented hydrolysate beer of between about 6% and 10%, the heat load requirement is based on the heat requirement of the beer column or that of the evaporation train. The water load, which is up to 3-fold higher than in a grain ethanol process, may be fully recycled in the present process at reasonable energy usage.

In one embodiment the rectification column temperature is run at the highest temperature in the heat integrated system with energy supplied to it from the power station. In one embodiment the rectification column is run with the temperature at the bottom being at least about 150° C. In various embodiments the rectification column is run with the temperature at the bottom being at least about 150° C., 155° C., 160° C., or greater.

In one embodiment, heat from the rectification column is used to heat the first effect evaporator, heat from first effect steam produced in the first effect evaporator is used to heat the second effect evaporator, heat from second effect steam produced in the second effect evaporator is used to heat the third effect evaporator, heat from third effect steam produced in the third effect evaporator is used to heat the fourth effect evaporator, and heat from fourth effect steam produced in the fourth effect evaporator is used to heat the beer column. In embodiments with more than four evaporation effects, steam is passed from one to the next in the same manner and the final effect may exhaust steam directly to the beer column. The beer column runs at the lowest temperature in the train, typically a pressure of 6 to 10 psia (41.4 to 68.9 kilopascal).

In one embodiment heat from the rectification column is used as a heat source on the first effect evaporator and steam produced in the first effect evaporator is passed either to a second effect evaporator or to a vapor recompression unit by which it is compressed and used as a heat source back on the first effect using a different heat exchange system to that used by the rectification column heat. If a large proportion of the steam generated in the first evaporation stage is returned as a heating medium to the first stage, then this first stage may operate as three or four effects of evaporation based on the heat load from the rectification column.

Steam in excess of that required on the first evaporation stage passes to a second stage of evaporation and can be used as a heating medium for it. Because the last stage of evaporation has a high boiling point elevation due to a high concentration of low volatility solutes, the last stage is less suitable for vapor recompression equipment. The steam generated in this final stage of evaporation may be injected into the base of the beer column as the heat source. Thus two stages of evaporation may operate with the capacity of four to six equivalent effects of evaporation because of the function served by the vapor recompression unit.

Overall, the evaporator train may require some additional heat to that required by the beer column, but the total heat required is near sustainability. If evaporators were run as in a typical grain ethanol process, the evaporator load would dominate the heat requirement. Thus the present heat integration process provides an economical use of heat for a cellulosic ethanol production process.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “kg” means kilograms; “hr” means hour; “ft” means feet.

### Example 1

### Effect of Running First Effect Evaporator at 105° C. On Fouling

Thin stillage was prepared by removing solids by filtering whole stillage that was produced in a cellulosic ethanol process. Corn stover was milled and pretreated with low concentration ammonia at a concentration of less than 12 weight %, then enzymatically saccharified with a consortium of cellulases and hemicellulases. The resulting hydrolysate was fermented using a Zymomonas mobilis ethanologen. The resulting beer was distilled to remove ethanol leaving whole stillage that was filtered to produce the thin stillage.

The thin stillage with a % total solids of 4.5% was concentrated to a syrup containing 50-55% solids through a series of evaporators, consisting of a 3-effect falling film evaporator and a forced circulation evaporator (Dedert Corporation, Homewood, Ill.). The thin stillage was fed to the 1^{st} stage evaporator with a flow rate ranging from 600-800 kg/hr. The three-effect evaporator operated at elevated pressures with the 3^{rd} effect vapor pressure controlled at 1.35 bar (135 kilopascal), while the forced circulation unit operated under vacuum at 0.85 bar (85 kilopascal). Steam at a pressure of 3.5-3.8 bar (350-380 kilopascal) was supplied to the 1^{st} effect evaporator while additional steam was injected to the forced circulation unit. Operating the forced circulation unit under vacuum not only minimized the decomposition risk of the high-solids syrup but also reduced the energy requirement.

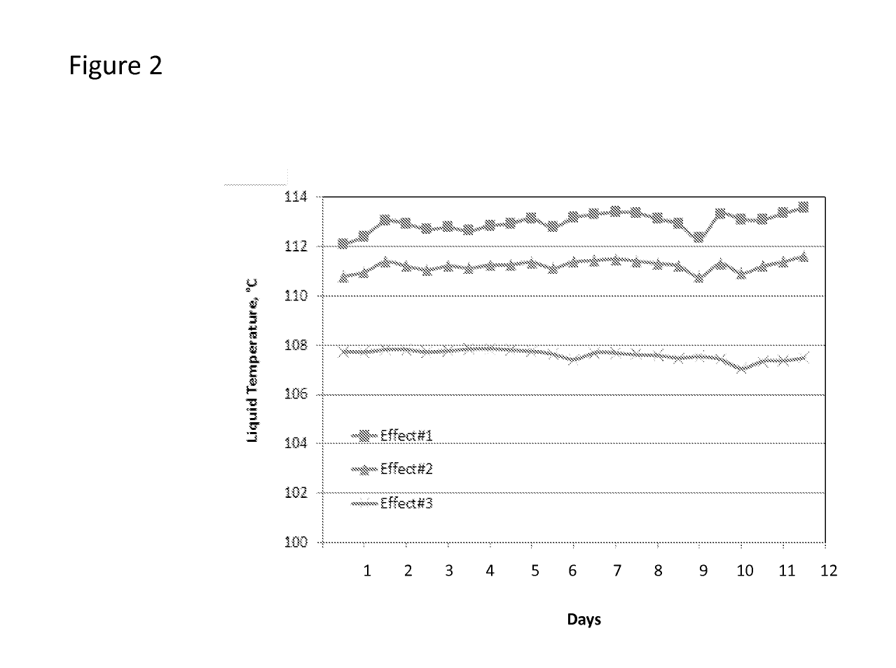

Temperatures of the evaporators were measured over 12 days. The liquid temperature of the first effect evaporator was around 113° C. All three evaporators ran above 105° C. as shown in FIG. 2.

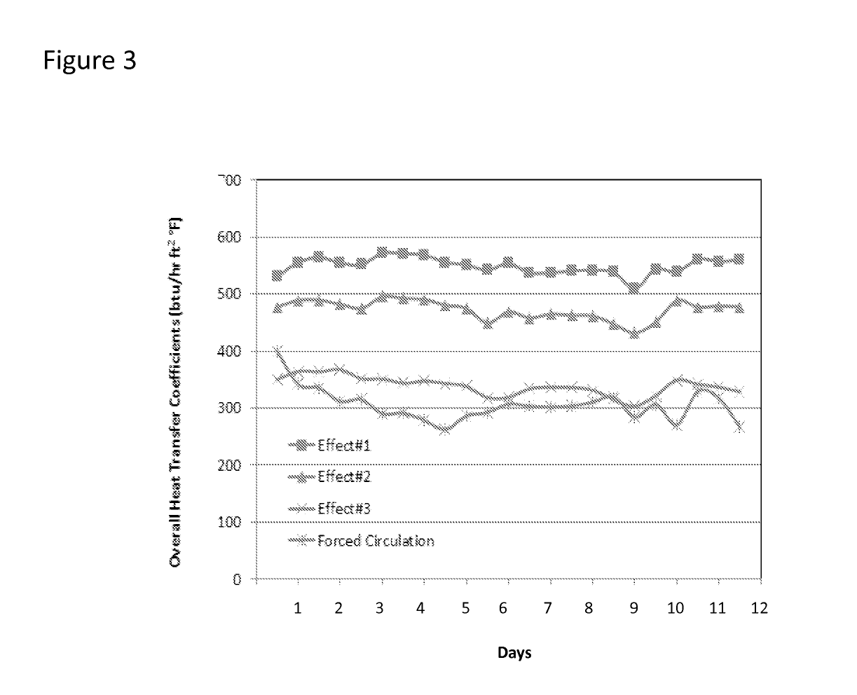

The overall heat transfer coefficient is calculated by dividing the heat transfer rate (in Btu) by the product of heat transfer surface area (in ft²) and log mean temperature difference (in ° F.). The heat transfer surface areas for each of the 3 falling film evaporators and the forced circulation evaporator were 164 ft² (15.2 meter²) and 221 ft² (20.5 meter²), respectively. The log mean temperature differences for the evaporators ranged from 4.5 to 8° F. The overall heat transfer coefficients for all four evaporators during the trial were calculated and are shown in FIG. 3.

When an evaporator surface is fouled, less heat is transferred from the steam to the process side, leading to less vaporization of the liquid medium and a lower overall heat transfer coefficient; as fouling occurs, the overall heat transfer coefficient trends downward with time. As shown in FIG. 3, the overall heat transfer coefficients for all stages of evaporators held fairly steady throughout the 11 day period, with the 1^{st} effect being the highest due to the lower % solids and the forced circulation heat exchanger being the lowest due to the high % solids. The % solids in the final syrup was controlled between 50% to 55%. Thus little to no sign of fouling was observed for all evaporators over an 11-day period of operation with the 1^{st} effect evaporator operating at above 105° C.

1. A process for the production of ethanol and recycle water comprising:
a) providing a hydrolysate fermentation medium;
b) fermenting the medium in a fermenter in the presence of a microorganism that produces ethanol to produce a beer comprising from about 6% to about 10% ethanol, water, solutes, and solids;
c) providing a distillation and water handling system comprising the elements:
I) a beer column;
II) a rectification column;
III) a solids removal mechanism; and
IV) an evaporation train comprising at least four evaporation effect equivalents;
wherein each element in the system is connected to at least one other element in the system, and wherein the beer column, the rectification column, and the evaporation train form a heat integrated system;
d) passing the beer into the beer column of (I) where a beer column ethanol-rich vapor stream is separated from a water stream containing solutes and solids;
e) condensing the beer column ethanol-rich vapor stream forming a beer column ethanol-rich stream;
f) passing the beer column ethanol-rich stream into the rectification column of (II) wherein a further ethanol-enriched rectification column vapor stream is separated from an ethanol depleted water stream;
g) passing the water stream containing solutes and solids of (d) through the solids removal mechanism of (III) producing a water stream containing solutes with solids substantially removed;
h) passing the water stream containing solutes with solids substantially removed of (g) through the evaporation train of (IV) producing an evaporate comprising water containing high-volatility solutes and a syrup comprising low-volatility solutes, wherein a first effect evaporator of the evaporator train of (IV) is run at a temperature that is greater than 105° C. for at least about 10 days without fouling of heat exchange surfaces; and
i) treating the evaporate of (h) to substantially remove the high-volatility solutes producing recycle water;
wherein ethanol-enriched vapor is produced and water is processed for recycle.

2. The process of claim 1 wherein the at least four evaporation effect equivalents comprises a series of at least four in-line separate evaporators in a multi-effect train, including at least a first effect evaporator, a second effect evaporator, a third effect evaporator, and a fourth effect evaporator.

3. The process of claim 2 wherein in the heat integrated system heat from the rectification column is used to heat the first effect evaporator, heat from first effect steam produced in the first effect evaporator is used to heat the second effect evaporator, heat from second effect steam produced in the second effect evaporator is used to heat the third effect evaporator, heat from third effect steam produced in the third effect evaporator is used to heat the fourth effect evaporator, and heat from fourth effect steam produced in the fourth effect evaporator is used to heat the beer column.

4. The process of claim 1 wherein the at least four evaporation effect equivalents comprises at least one first evaporator stage and at least one vapor recompression unit.

5. The process of claim 4 wherein the at least four evaporation effect equivalents comprises at least two evaporator stages in a multi-effect train and at least one vapor recompression unit.

6. The process of claim 4 or 5 wherein in the heat integrated system heat from the rectification column is used to heat the first evaporator stage, a first portion of heat from steam produced in the first evaporator stage is passed through the vapor recompression unit and back to the first evaporator stage, a second portion of heat from the steam produced in the first evaporator stage is passed either to a second evaporator stage if present, and heat from steam produced in the second stage evaporator is passed to the beer column, or directly to the beer column.

7. The process of claim 1 wherein the rectification column temperature is the highest temperature in the heat integrated system.

8. The process of claim 7 wherein the rectification column is run with the temperature at the bottom being at least about 150° C.

9. The process of claim 1 wherein the most abundant high-volatility solute in the evaporate of (h) is acetic acid.

10. The process of claim 1 wherein treating the evaporate in (h) is by a process selected from anaerobic digestion, aerobic digestion, nanofiltration, ultrafiltration, reverse osmosis membrane separation, and ion exchange separation.

11. The process of claim 1 wherein at least a portion of the recycle water of (i) is used in the hydrolysate fermentation medium.

12. The process of claim 1 wherein the further ethanol-enriched rectification column vapor stream is passed through a molecular sieve producing a further purified ethanol product and a molecular sieve stream comprising water and ethanol.

13. The process of claim 12 wherein the molecular sieve stream is passed to the beer column or the rectification column.

14. The process of claim 1 wherein a stream containing water vapor and fusel oils is collected from the rectification column and passed through the molecular sieve, wherein the fusel oils are combined with the further purified ethanol product.

15. The process of claim 1 wherein the cellulosic biomass is selected from the group consisting of corn cobs, crop residues such as corn husks, corn stover, grasses, wheat straw, barley straw, hay, rice straw, switchgrass, waste paper, sugar cane bagasse, sorghum plant material, soybean plant material, components obtained from milling of grains, trees, branches, roots, leaves, wood chips, sawdust, shrubs and bushes, vegetables, fruits, and flowers.

16. The process of claim 1 wherein the microorganism is an ethanologen selected from the group consisting of a yeast and a bacterium.

17. The process of claim 16 wherein the ethanologen is selected from the group consisting of Zymomonas, Zymobacter, and Saccharomyces.

18. The process of claim 1 wherein the concentration of ethanol in the beer is between about 7% and about 9%.
